(19) United States
(12) Patent Application Publication
(10) Pub. No.: US 2023/0248998 A1
(43) Pub. Date: Aug. 10, 2023

(54) SYSTEM AND METHOD FOR PREDICTING DISEASES IN ITS EARLY PHASE USING ARTIFICIAL INTELLIGENCE

(71) Applicants: Buvaneswari Natarajan, Edison, NJ (US); S. Bose, Tamil Nadu (IN); Poongodi Manoharan, Doha (QA)

(72) Inventors: Buvaneswari Natarajan, Edison, NJ (US); S. Bose, Tamil Nadu (IN); Poongodi Manoharan, Doha (QA)

(21) Appl. No.: 18/299,670

(22) Filed: Apr. 12, 2023

(51) Int. Cl. A61N 5/10; G06T 7/00; G06T 3/40; G06T 7/11; G06T 7/194; G06T 5/00; G06T 5/10; G16B 25/10; G16H 30/40; G16H 15/00; G16H 10/60

(52) U.S. Cl. CPC A61N 5/1039; G06T 3/40; G06T 5/002; G06T 5/10; G06T 7/11; G06T 7/0012; G06T 7/194; G16B 25/10; G16H 10/60; G16H 15/00; G16H 30/40; G06T 2207/10024; G06T 2207/10081; G06T 2207/10088; G06T 2207/10132; G06T 2207/20081; G06T 2207/20132; G06T 2207/30081; G06T 2207/30096; G06T 2207/30104

(57) ABSTRACT

The system comprises an image acquisition device for collecting medical images; an image pre-processing device for enhancing the visual quality; an image segmentation device for extracting the region of interest from the image’s background by identifying each image’s pixel characteristics, and dividing the image into segments; a feature extraction and selection device for extracting a set of features and selecting the optimized features; a model training device for training a fuzzy logic-based prediction model and a plurality of diagnosis-specific treatment response models to predict treatment response; and a central processing device coupled to a user input device for receiving a subject patient dataset including features obtained for a reduced feature dataset and comparing the subject patient dataset to a feature data scheme for predicting a response for the subject patient thereby predicting the diseases in its early stage.

### FIELD OF THE INVENTION

The present disclosure relates to the field of patient diagnosis, monitoring and treatment, and more particularly, to a system and method for facilitating early detection of diseases along with detection of type and stage of disease using artificial intelligence.

### BACKGROUND OF THE INVENTION

Numerous medications have been developed in modern medicine to treat a variety of ailments. However, the treating medical professional (e.g., doctor, nurse, nurse practitioner, etc.) must ensure that the patient receives the best possible medical care for the condition they are experiencing. requires the patient to provide data on a variety of parameters. For reasons like the patient being unconscious, unable to express their symptoms, not knowing all of the information, etc., oral recitation by the patient is not always the best data source. As a result, a different method of gathering this information from the patient is required. As a result, a wide range of medical instruments, such as thermometers, sphygmomanometers, and stethoscopes, have been developed to facilitate the collection of patient data by medical professionals.

Measurements of a relevant set of biomarkers serve as the basis for health assessment and diagnosis of particular diseases. The diagnosis, severity, and course of a disease are all aspects of a health assessment. Various sensors are used to measure vital signs like pulse rate, temperature, respiration rate, and blood pressure. These measurements are taken once or over a long period of time, either continuously or intermittently. A fever diagnosis, for instance, can be made with just one temperature reading, but a hypertension diagnosis requires at least three blood pressure readings taken at least a week apart. For obstructive sleep apnoea to be diagnosed, the patient must have their heart, lungs, and brain activity, breathing patterns, arm and leg movements, and blood oxygen levels continuously measured for at least four hours while they are asleep.

Artificial Intelligence, or AI, has become an important tool for making decisions and making predictions in a variety of fields over the past few years. The widespread use of smart homes, virtual assistants, speakers, smart marketing, unmanned driving, autonomous driving, unmanned aerial vehicles, robots, smart medical services, and smart customer service are just a few examples. It is anticipated that the application of artificial intelligence technology will expand as technology advances and assume increasingly significant values.

As of now, occasion choice or expectation in the clinical field is acknowledged by joining an information chart with a brain network model in AI and such. In particular, highlight learning is done on the information diagram of the illness class to get substance vectors, connection vectors and other low-layered vectors, then the low-layered vectors are brought into a brain network model to understand a specific occasion choice model, and occasion choice is finished in light of the model and current information. or combining the disease class knowledge map feature learning with the objective function of the technique, using an end-to-end method to perform the joint learning of the technique model, feeding the supervision signal in the final technique model back to the knowledge map feature learning in real time, continuously adjusting, achieving a particular event decision model, and completing the event decision.

However, there is only one processing of the knowledge graph, and the technique model is only connected to the manpower and material resources labelled knowledge graph. In the view of the forgoing discussion, it is clearly portrayed that there is a need to have a predictive system and method using artificial intelligence.

### SUMMARY OF THE INVENTION

The present disclosure seeks to provide an intelligent system and method using artificial intelligence for detecting multiple diseases using digital images and providing a treatment plan for the same.

In an embodiment, a system for predicting diseases in its early phase using artificial intelligence is disclosed. The system includes an image acquisition device for collecting medical images in digital format from a plurality of medical prediction centers and a plurality of medical record databases, wherein the collected images are typically captured using one of both of a general-purpose camera or real-time image capturing tools such as CT scan, radiology, MRI, Ultrasound, and nuclear medicine imaging.

The system further includes an image pre-processing device for enhancing the visual quality of an image by reducing noises and identifying the image’s texture, color, and shape to produce a clean image, wherein the image pre-processing device comprising resizing images to lower pixel resolution to reduce the processing time and cropping images to remove unnecessary area and retaining the area of interest thereby eliminating the noise using filters followed by transforming the original RGB color to grayscale intensity to remove undesired variations in color.

The system further includes an image segmentation device for extracting the region of interest from the image’s background by identifying each image’s pixel characteristics, and dividing the image into segments consisting of similar characteristic pixels.

The system further includes a feature extraction and selection device for extracting a set of features selected from Asymmetry index, Entropy, Autocorrelation, Homogeneity, and Contrast used for the classification stage from the region of interest of the image and selecting the optimized features from the set of features.

The system further includes a model training device for training a fuzzy logic-based prediction model and a plurality of diagnosis-specific treatment response models to predict treatment response using an artificial intelligence and storing in a cloud server platform.

The system further includes a central processing device coupled to a user input device for receiving a subject patient dataset including features obtained for a reduced feature dataset and comparing the subject patient dataset to a feature data scheme for predicting a response for the subject patient, wherein comparing the subject patient dataset comprising determining a subject patient diagnosis of one of the known disorders indicated for the subject patient by the subject patient dataset upon deploying the prediction model to the subject patient dataset and applying the diagnosis-specific treatment response models to the subject patient dataset for predicting the response for the subject patient and predicting the diseases in its early stage, wherein the central processing device is configured to generate a medical report along with severity of the disease and stage of the disease.

In another embodiment, a method for predicting diseases in its early phase using artificial intelligence is disclosed. The method includes collecting medical images in digital format from a plurality of medical prediction centers and a plurality of medical record databases using an image acquisition device, wherein the collected images are typically captured using one of both of a general-purpose camera or real-time image capturing tools such as CT scan, radiology, MRI, Ultrasound, and nuclear medicine imaging.

The method further includes enhancing the visual quality of an image by reducing noises and identifying the image’s texture, color, and shape to produce a clean image through an image pre-processing device, wherein the image pre-processing device comprising resizing images to lower pixel resolution to reduce the processing time and cropping images to remove unnecessary area and retaining the area of interest thereby eliminating the noise using filters followed by transforming the original RGB color to grayscale intensity to remove undesired variations in color.

The method further includes extracting the region of interest from the image’s background by identifying each image’s pixel characteristics, and dividing the image into segments consisting of similar characteristic pixels by employing an image segmentation device.

The method further includes extracting a set of features selected from Asymmetry index, Entropy, Autocorrelation, Homogeneity, and Contrast used for the classification stage from the region of interest of the image and selecting the optimized features from the set of features using a feature extraction and selection device.

The method further includes training a fuzzy logic-based prediction model and a plurality of diagnosis-specific treatment response models to predict treatment response using an artificial intelligence and storing in a cloud server platform by deploying a model training device, wherein the fuzzy logic-based prediction model comprises: a fuzzifier for converting the medical images input into the fuzzy values; an inference engine for processing the fuzzy value by the reasoning engine employing a set of rules act as a set of rules to the cognitive content; a knowledgebase consists of rules, structured and unstructured information also named the database; and a de-fuzzifier for defuzzification of the fuzzy value upon changing the output from the logical thinking engine into medical images.

The method further includes receiving a subject patient dataset including features obtained for a reduced feature dataset via a user input device and comparing the subject patient dataset to a feature data scheme for predicting a response for the subject patient using a central processing device, wherein comparing the subject patient dataset comprising determining a subject patient diagnosis of one of the known disorders indicated for the subject patient by the subject patient dataset upon deploying the prediction model to the subject patient dataset and applying the diagnosis-specific treatment response models to the subject patient dataset for predicting the response for the subject patient and predicting the diseases in its early stage, wherein the central processing device is configured to generate a medical report along with severity of the disease and stage of the disease.

An object of the present disclosure is to detect and monitor multiple diseases using digital images.

Another object of the present disclosure is to detect the diseases in its early stage.

Another object of the present disclosure is to generate a medical report along with severity of the disease and stage of the disease.

Another object of the present disclosure is to provide a radiotherapy dose distribution upon receiving anatomical data of a human subject.

Yet another object of the present invention is to deliver an expeditious and cost-effective system for predicting diseases in its early phase using artificial intelligence.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a system for predicting diseases in its early phase using artificial intelligence in accordance with an embodiment of the present disclosure;

FIG. 2 illustrates a flow chart of a method for predicting diseases in its early phase using artificial intelligence;

FIG. 3 illustrates a flow chart of the Fuzzy logic process;

FIG. 4 illustrates a machine learning system; and

FIG. 5 illustrates a system with remote or central data/signal processing.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises...a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

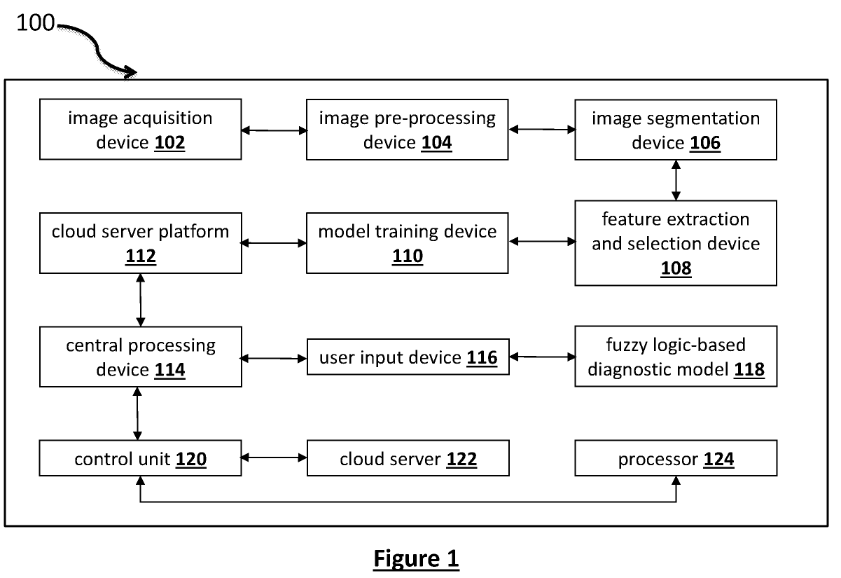

Referring to FIG. 1, a block diagram of a system for predicting diseases in its early phase using artificial intelligence is illustrated in accordance with an embodiment of the present disclosure. The system 100 includes an image acquisition device 102 for collecting medical images in digital format from a plurality of medical prediction centers and a plurality of medical record databases, wherein the collected images are typically captured using one of both of a general-purpose camera or real-time image capturing tools such as CT scan, radiology, MRI, Ultrasound, and nuclear medicine imaging.

In an embodiment, an image pre-processing device 104 is coupled to the image acquisition device 102 for enhancing the visual quality of an image by reducing noises and identifying the image’s texture, color, and shape to produce a clean image, wherein the image pre-processing device 104 comprising resizing images to lower pixel resolution to reduce the processing time and cropping images to remove unnecessary area and retaining the area of interest thereby eliminating the noise using filters followed by transforming the original RGB color to grayscale intensity to remove undesired variations in color.

In an embodiment, an image segmentation device 106 is coupled to the image pre-processing device 104 for extracting the region of interest from the image’s background by identifying each image’s pixel characteristics, and dividing the image into segments consisting of similar characteristic pixels.

In an embodiment, a feature extraction and selection device 108 is coupled to the image segmentation device 106 for extracting a set of features selected from Asymmetry index, Entropy, Autocorrelation, Homogeneity, and Contrast used for the classification stage from the region of interest of the image and selecting the optimized features from the set of features.

In an embodiment, a model training device 110 is coupled to the feature extraction and selection device 108 for training a fuzzy logic-based prediction model 118 and a plurality of diagnosis-specific treatment response models to predict treatment response using an artificial intelligence and storing in a cloud server platform 112.

In an embodiment, a central processing device 114 is coupled to a user input device 116 for receiving a subject patient dataset including features obtained for a reduced feature dataset and comparing the subject patient dataset to a feature data scheme for predicting a response for the subject patient, wherein comparing the subject patient dataset comprising determining a subject patient diagnosis of one of the known disorders indicated for the subject patient by the subject patient dataset upon deploying the prediction model 118 to the subject patient dataset and applying the diagnosis-specific treatment response models to the subject patient dataset for predicting the response for the subject patient and predicting the diseases in its early stage, wherein the central processing device 114 is configured to generate a medical report along with severity of the disease and stage of the disease.

In another embodiment, the prediction model 118 is employed for determining a diagnosis of a plurality of known disorders indicated by individual patient dataset and the plurality of diagnosis-specific treatment response models corresponding to a specific diagnosis of the known disorders, the treatment response models configured to use feature data to predict treatment response, wherein the prediction model is configured to: secure a three-layered ovary picture of a subject through clinical imaging gear, and performing picture denoising and upgrading treatment, wherein the image denoising enhancement process of the biomarker-based ovarian cancer assessment method comprising of completing essential denoising treatment on the first three-layered ovary picture to get an essential denoising picture and calculating the residual quantity of a central pixel for each unit region on the original three-dimensional ovary image using the respective numerical values of the specific energy parameters for the initial denoising image and the three-dimensional ovary image. Then, compare the image to the position of the ovarian tumor after the enhancement treatment. Then, utilize a medical instrument to measure the concentration of at least one small molecule biomarker in an ovarian cancer tumor of a subject. Then, compare a control sample to the concentration of the small molecule biomarker that was obtained, wherein in the event that the convergence of the little sub-atomic biomarkers surpasses or is lower than a relating limit esteem, getting CA125 information, HE4 information and Dad information of a serum test to be distinguished of the subject by utilizing an ID program. Then, utilize the CA125, HE4, and PA data to calculate an area value under a working characteristic curve. Thereafter, use an evaluation program based on the concentration of the small molecule biomarker, the CA125 data, the HE4 data, and the PA data from the serum sample, as well as the area value under the working characteristic curve, evaluating the subject’s ovarian cancer condition and producing an evaluation report for a doctor to diagnose and select a treatment mode.

In another embodiment, the detection of the concentration of at least one small molecule biomarker in the ovarian cancer tumor is accomplished using the biomarker-based ovarian cancer assessment method, wherein the biomarker-based ovarian cancer assessment method comprises: obtaining a sample from the subject, chosen from the blood, serum, and plasma categories; the little atom biomarker is chosen from the gathering comprising of: hydroxy acids, adipic acid, hydroxybutyric acid, and ketone bodies; dihydroxybutyric acid; trihydroxybutyric acid; detecting the ovarian cancer-specific small molecule biomarker by contacting the sample with an antibody or antigen-binding fragment that is capable of specifically binding to it; reading a decile value from the frequency profile of concentrations of the small molecule biomarker and comparing the determined concentration of the small molecule biomarker to the reference frequency profile of concentrations of the small molecule biomarker.

In another embodiment, the fuzzy logic-based prediction model 118 comprises a fuzzifier for converting the medical images input into the fuzzy values. In one embodiment, an inference engine is connected to the fuzzifier for processing the fuzzy value by the reasoning engine employing a set of rules act as a set of rules to the cognitive content. In one embodiment, a knowledgebase consists of rules, structured and unstructured information also named the database. In one embodiment, a de-fuzzifier is used for defuzzification of the fuzzy value upon changing the output from the logical thinking engine into medical images.

In another embodiment, the image is resized to have a fixed pixel using an image scaling technique such as normalization, and the image’s color space transformation techniques are used to transform the original RGB color to grayscale intensity to remove undesired variations in color, wherein the contrast enhancement technique is used to sharpen the border of the images and improve the brightness between the foreground and background of the image, wherein the degraded image is recovered from a blurred and noisy image in the image restoration, wherein a plurality of filtering techniques are used to de-noise or suppress and smoothen the image, selected from Median filter, Adaptive median filter and to restore the image from blur, which is caused due to the poor focusing of the camera, wherein restoration is performed by using filters preferably a Gaussian filter, wherein the images are smoothened using an image restoration filter, and the image still contain artifacts or other noises, which are removed using various methods such as Curvilinear structure detection, Mathematical morphology, Top Hat transform, Bottom Hat transform, Dull Razor, and Gabor filter.

In another embodiment, a control unit 120 is equipped with the artificial intelligence for generating the feature data scheme, wherein the control unit 120 includes a cloud server 122 for storing a first-level training dataset that contains records with measured patient-related data from a lot of patients, including clinical and/or laboratory data, diagnoses of the presence or absence of known disorders, and information on patient treatment responses, wherein the first-level training dataset further includes one or more of markers are selected from the group comprising following component : Blood Hemoglobin concentration (HbC), transferrins, kreatinin, blood platelet, low-density lipoprotein (LDL), albumin, total protein and calcium.

In another embodiment, the control unit 120 further includes a processor 124 for processing the measured patient-related data to extract features using the measured patient-related data to build an extracted feature dataset and generating the feature data scheme by processing the extracted feature dataset thereby processing the data to produce characteristics that seemed to discriminate for an effective prediction, resulting in the reduced feature dataset, wherein the feature data strategy includes a reduced feature dataset with a lower cardinality than the extracted feature dataset, wherein the individual Z score of each marker Mi is determined by following formula, where ME (i, j) is the subject’s individual average value, VAR (i, j) is the subject’s individual variance, and Mi tables show the value of one of the described markers at time i. (2.3) The weighting function is then used to combine each individual Z score. The weighting function is derived from plasma volume, which is the known variation of each relevant marker, and the consistency between all Z scores. Blood Starch is the estimated value of the capacity variation when using the Z score.

In another embodiment, the processor 124 is configured to cause the system to determine the medical images through the first recognition model to generate the lesion recognition report used for indicating whether the medical images comprises the lesion, the processor 124 is configured to cause the apparatus to search the medical images for a lesion feature by using the artificial intelligence, wherein the lesion feature being a second image feature obtained by learning a first medical image set of a normal organ and a second medical image set of an organ having a lesion by the deep learning network during training to generate the lesion recognition report according to a second searching report, and the lesion feature existing in the second medical image set and not in the first medical image set.

In another embodiment, a feature response of the lesion feature of the first lesion degree in the digital image having a lesion degree lower than the first lesion degree, which is less than a threshold, wherein the lesion degree recognition report of the medical images further comprises a lesion degree label of the medical images and the lesion degree label of the medical images comprises a first recognition report of an image block having a severe lesion degree in image blocks segmented from the medical images, a second recognition report of a lesion degree of the medical images determined using feature information of all the image blocks, and a comprehensive report determined using the first and second recognition report.

In another embodiment, the stage is preferably defined from 0-5, wherein 0 indicates perfectly fine and 5 is a worst case, that may require serious surgery, wherein the central processing unit, using the artificial intelligence prescribes a treatment plan according to the stage and type of the disease, wherein the diseases includes skin diseases, liver diseases, heart diseases, Alzheimer, cancer and the like, wherein the biomarker-based ovarian cancer assessment method is defined by the fact that an identification procedure is used to obtain the CA125, HE4, and PA data of the subject’s serum sample in the event that a small molecule biomarker selected from the group consisting of hydroxyacids and adipic acid is increased in comparison to a control.

In another embodiment, an exemplary treatment plan, in case cancer, provides a radiotherapy dose distribution upon receiving anatomical data of a human subject and generating a radiotherapy dose data corresponding to the mapping thereby converting the radiotherapy dose data from the generative model into a radiotherapy dose distribution followed by outputting the radiotherapy dose distribution for use in the radiotherapy treatment of the human subject, wherein the anatomical data indicating a mapping of an anatomical area for radiotherapy treatment of the human subject, and wherein the radiotherapy dose data from the generative model identifies radiotherapy dosage to be delivered to the anatomical area.

In another embodiment, the prediction of prostate carcinogenesis and metastasis comprises taking a three-dimensional image of a person’s prostate and bladder and selecting a layer in a sagittal image that passes through the bottom of the bladder thereby obtaining a cross-sectional image at the layer, followed by identifying the fat outline and the prostate outline around the prostate in the cross-sectional image, which calculates the fat area around the prostate (PPFA) based on the area in the fat outline around the prostate, wherein the proportion PPFA/Dad of the region of the fat around the prostate to the region of the prostate, and the gamble worth of the event and the metastasis of the prostate malignant growth is in direct extent to the proportion PPFA/Dad, wherein the central processing device uses a formula based on an age variable, a rectal index variable, a family genetic history variable, a prostate image report and a data system scoring variable, a PSA value variable, and a ratio variable of a peripheral fat area of the prostate and a prostate area to calculate a risk value for the first diagnosis of prostate cancer, wherein the output device then displays the risk value for the first diagnosis of prostate cancer, wherein the formula is as follows:

$$\begin{matrix}L o g i t ( P ) = \\ I n ( P / ( 1 - P ) ) = \\ 1.037 * A g e + c o e f D R E + c o e f H i s t o r y + 1.033 * P S A + c o e f P I R A D S + 1.066 * ( P P F A / P A ) .\end{matrix}$$

In another embodiment, the prediction of prostate cancer’s occurrence and metastasis comprises: the handling gadget is utilized for diagnosing lymph hub metastasis probability factors, prostate picture reports and information framework scoring factors, proportion factors of fat region around the prostate and prostate region, Gleason scoring factors, obsessive T stage factors, public service announcement esteem factors and Ki-67 articulation level factors as indicated by X-ray before an activity, working out to get a lymph hub metastasis risk worth of a prostate malignant growth patient as per a recipe, and yielding the lymph hub metastasis risk worth of the prostate disease patient by the result gadget, wherein the equation is as per the following: Logit(P)=In(P/(1-P))=coefPre-LNM+coefPIRADS+coefRatio+coefpT-stage+1.008*PSA+1.152*Ki-67, where P is the predicted value of prostate cancer’s lymph node metastasis risk, coefPre-LNM is the possibility of lymph node metastasis diagnosed prior to MRI surgery, coefPIRADS.

In another embodiment, for the purpose of predicting the occurrence of prostate cancer, an age parameter, a rectal index parameter, a family genetic history parameter, a PSA value parameter, and a PIRADS scoring parameter are combined with the ratio PPFA/PA of the area of the fat surrounding the prostate to the area of the prostate.

In another embodiment, the fuzzy logic-based prediction model involves using the Dopplerographic method to measure quantitative blood flow indicators, wherein the maximum systolic speed and resistance index are assessed at the level of the interlobar renal arteries before and 30 minutes after an intramuscular injection of lasix at a rate of 1 mg/kg and patients with a final diastolic rate decrease of more than 5% and an increase in resistance index of more than 2% are diagnosed with a normal response.

In another embodiment, the fuzzy logic-based prediction model employs at least two types of cancer-related proteins in a sample obtained from a subject having cancer as a prognostic indicator of cancer by identifying at least two types of cancer-associated proteins in the sample from the subject and quantifying the at least two cancer-associated proteins in the sample thereby normalizing the at least two cancer-related proteins in the sample to obtain a normalized value for each cancer-related protein in the sample followed by obtaining a biomarker index and comparing the normalized value of the first cancer-related protein adding a technique, wherein the carcinoma is selected from the group consisting of breast, lung, prostate, colon, liver, thyroid, kidney, and bile duct carcinomas.

In another embodiment, a tumor antigen selected from the following group is present in at least one of the two types of cancer-related proteins: AKT; p-AKT; CA150, Tn antigen in the blood; CA19. -9; CA50; CAB39L; CD22; CD24; CD63; CD66e, CD66a, CD66c, and CD66d; CTAG1B; CTAG2; Antigen oncofetal (CEA); EBAG9; EGFR; FLJ14868; FMNL1; GAGE1; GPA33; LRIG3; lung cancer, group two; MAGE1, M2A tumor fetal antigen MAGEA10; MAGEA11; MAGEA12; MAGEA2; MAGEA4; MAGEB1; MAGEB2; MAGE 3; MAGEB4; MAGEB6; MAGE1; MAGE1; MAGEH1; MAGE2; MGEA5; Protein kinase MOK; MAPK; p-MAPK; mTOR; p-mTOR; MUC16; MUC4; antigen related to melanoma; OCIAD1; OIP5; ovarian malignant growth related antigen; PAGE4; PCNA; PRAME; plastin L; prostate mucin antigen (PMA); antigen specific for prostate (PSA); PTEN; RASD2; ROPN1; SART2; SART3; SPANXB1; SSX5; STEAP4; STK31; TAG72; TEM1; XAGE2; 1-fetoprotein, a Wilms tumor protein; and original tumor antitumor of epithelial origin. The method of claim 1, in which at least one of the two types of cancer-associated proteins includes a tumor-associated antigen from one of the following groups: 5T4; AKT; p-AKT; ACRBP; blood bunch Tn. CD164; CD20; CTHRC1; ErbB2; FATE1; HER2; HER3; GPNMB; Galectin8; HORMAD1; LYK5; MAGEA6; MAGEA8; MAGEA9; MelanA; gp100 melanoma; NYS48; PARP9; PATE; prostein; PTEN; SDCCAG8; SEPT1; SLC45A2; TBC1D2; TRP1; XAGE1, wherein the cancer is selected from the group consisting of Adrenal tumors, bile duct cancer, bladder cancer, bone cancer, brain tumors, breast cancer, heart sarcoma, cervical cancer, colorectal cancer, uterine Endometrial cancer, esophageal cancer, germ cell cancer, gynecological cancer, head and neck cancer, hepatoblastoma, kidney cancer, pharyngeal cancer, leukemia, liver cancer, lung cancer, lymphoma, melanoma, multiple myeloma, neuroblast Cell tumor, oral cancer, ovarian cancer, pancreatic cancer, parathyroid cancer, pituitary tumor, prostate cancer, retinoblastoma, rhabdomyosarcoma, skin cancer (non-melanoma), stomach (digestive organ) cancer, testicular cancer Thyroid cancer, uterine cancer, vaginal cancer, vulvar cancer, and Wilms tumor.

In another embodiment, the artificial intelligence is offered for the cancer-associated protein to serve as a marker for the presence of cancer in the subject upon discovering the presence of a first cancer-related protein in a biological sample taken from the individual, which may be PTEN, p-AKT, p-mTOR, p-MAPK, EGFR, HER2, HER3, or a combination of two or more of these proteins and determining the first cancer-associated protein’s degree of protein expression thereby comparing the first cancer-associated protein’s protein expression level in the biological sample to a predetermined statistically significant cutoff value, where non-cancerous changes in the first cancer-associated protein’s protein expression levels in the biological sample compared to the sample indicate the presence of cancer in the subject.

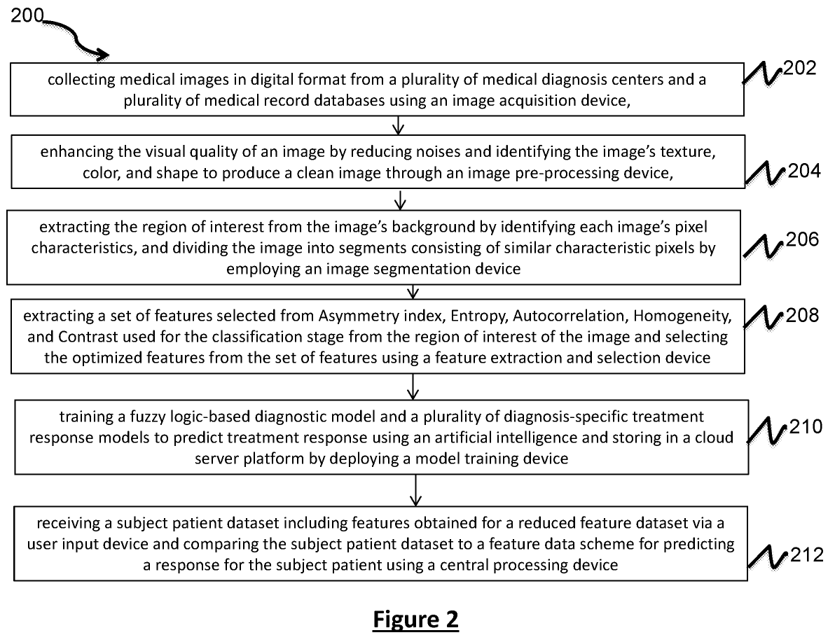

FIG. 2 illustrates a flow chart of a method for predicting diseases in its early phase using artificial intelligence. At step 202, method 200 includes collecting medical images in digital format from a plurality of medical prediction centers and a plurality of medical record databases using an image acquisition device 102, wherein the collected images are typically captured using one of both of a general-purpose camera or real-time image capturing tools such as CT scan, radiology, MRI, Ultrasound, and nuclear medicine imaging.

At step 204, method 200 includes enhancing the visual quality of an image by reducing noises and identifying the image’s texture, color, and shape to produce a clean image through an image pre-processing device 104, wherein the image pre-processing device 104 comprising resizing images to lower pixel resolution to reduce the processing time and cropping images to remove unnecessary area and retaining the area of interest thereby eliminating the noise using filters followed by transforming the original RGB color to grayscale intensity to remove undesired variations in color.

At step 206, method 200 includes extracting the region of interest from the image’s background by identifying each image’s pixel characteristics, and dividing the image into segments consisting of similar characteristic pixels by employing an image segmentation device 106.

At step 208, method 200 includes extracting a set of features selected from Asymmetry index, Entropy, Autocorrelation, Homogeneity, and Contrast used for the classification stage from the region of interest of the image and selecting the optimized features from the set of features using a feature extraction and selection device 108.

At step 210, method 200 includes training a fuzzy logic-based prediction model 118 and a plurality of diagnosis-specific treatment response models to predict treatment response using an artificial intelligence and storing in a cloud server platform 112 by deploying a model training device 110.

At step 212, method 200 includes receiving a subject patient dataset including features obtained for a reduced feature dataset via a user input device 116 and comparing the subject patient dataset to a feature data scheme for predicting a response for the subject patient using a central processing device 114, wherein comparing the subject patient dataset comprising determining a subject patient diagnosis of one of the known disorders indicated for the subject patient by the subject patient dataset upon deploying the prediction model 118 to the subject patient dataset and applying the diagnosis-specific treatment response models to the subject patient dataset for predicting the response for the subject patient and predicting the diseases in its early stage, wherein the central processing device 114 is configured to generate a medical report along with severity of the disease and stage of the disease.

In one embodiment, an in vitro method for diagnosing a patient’s tumor disease using diagnosis-specific treatment response models comprising steps of i) finding an IVD marker or IVD marker panel with a relatively high sensitivity to the tumor disease in at least one patient biological sample; ii) figuring out how many patients tested positive because of a modified reference range for the IVD marker or IVD marker panel, where the modified reference range is one that is adjusted so that a certain number of people who have false negative tests, a certain number of people who have false positive tests, and a certain number of people who will eventually need to be subjected to imaging diagnostics to clarify false negative and false positive results are balanced in relation to one another so that tumor screening may be possible; and iii) deciding to use an imaging technique specific to the tumor disease so that at least one of the possible false negative and false positive IVD results can be clarified; or performing an imaging technique to image the tumor, or repeating (i) and (ii) after a predetermined time period.

In one embodiment, the biological sample is selected from a blood sample, a serum sample, a plasma sample, a urine sample, a fecal sample, a saliva sample, a spinal fluid sample, a nasal discharge sample, a sputum sample, a bronchoalveolar lavage sample, a semen sample, a breast discharge sample, a wound discharge sample, an ascites sample, a gastric juice sample or a sweat sample.

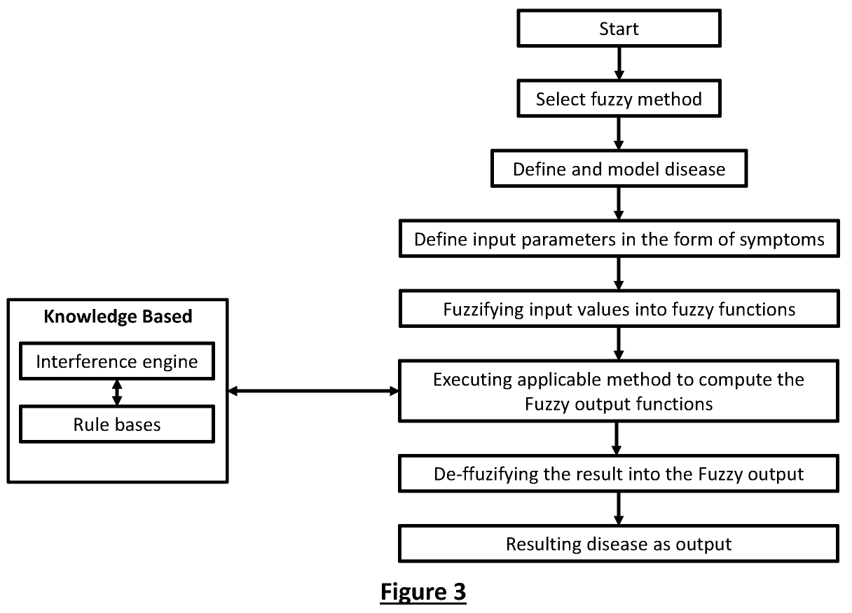

FIG. 3 illustrates a flow chart of the Fuzzy logic process. A form of many-valued logic is logic in which the actual value of variables can be expressed in decimal or any complex number between zero and one for each complete. The subsequence steps typically produce the fuzzy logic process for disease identification depicted in FIG. 3.

1) Fuzzifier: The Fuzzification method is finish by a Fuzzifier. It is a method of adjusting a crisp input worth to the fuzzy set. Therefore, Fuzzifier is employed as a mapping from observant input to fuzzy value.

2)Inference engine: When finishing the fuzzification method, fuzzy value processed by the reasoning engine employing a set of rules act as a set of rules to the cognitive content.

3)Knowledgebase: This is the main component of the fuzzy logic system. The overall fuzzy system depends on the cognitive content. Basically, it consists of rules, structured and unstructured information also named the database.

4) De-fuzzifier: The method of changing the output from the logical thinking engine into crisp logic. Fuzzy value is associate input to the defuzzification that fuzzy value.

When it comes to achieving intelligent behavior through the creation of fuzzy categories for a few parameters, fuzzy logic is one of the AI techniques that is taken into consideration. Humans are capable of comprehending the principles and criteria. A site professional largely defines these rules and the fuzzy categories. Mathematical logic necessitates extensive human intervention as a result. The specific course of data fundamentally gives a show of the data in fluffy rationale. In the medical field, machine learning can even perform one of these representations much more effectively than fuzzy logic. The estimation statistical model cannot deliver satisfactory performance results. Large data values, missing values, and categorical data are all missed by statistical models. Machine learning (ML) can be used to achieve all of the aforementioned goals. ML assumes a fundamental part in various applications, for example, regular language handling, data mining, picture location, and illness discovery. In each of the aforementioned domains, ML offers problem-specific solutions. As a result, ML also makes it easier for advanced healthcare diagnosis and treatment options.

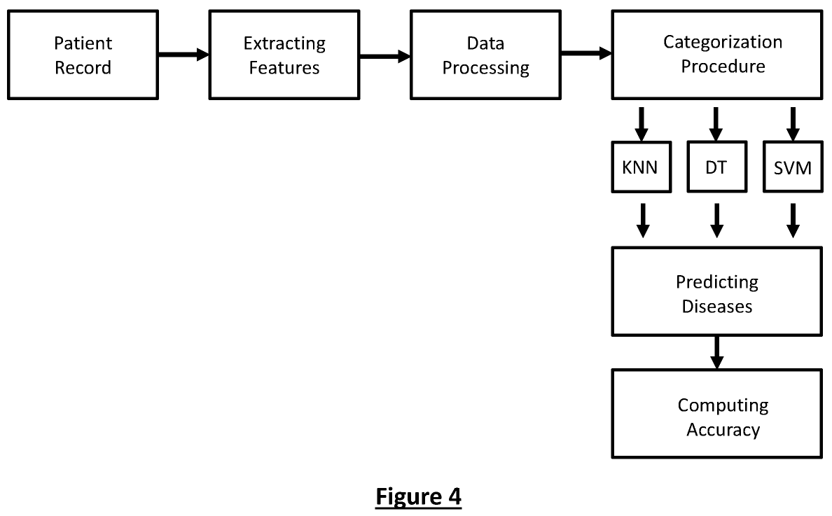

FIG. 4 illustrates a machine learning system. Techniques for supervised learning are used in machine learning. These techniques look for patterns in the data and make better decisions. The key objective is to allow the machines to advance naturally without human impedance and change the reaction as needs be. Predicting certain chronic diseases like kidney disease, diabetes, heart disease, breast cancer, and lung conditions, among others, is our primary focus. Computer systems now have new capabilities that can never been imagined. A subfield of artificial intelligence known as “machine learning” empowers machines to learn from examples in order to examine how various models perform in ML without the use of human judgment. Data Collection provides a step-by-step explanation of how ML works: The gathering of data is the very first step. Because both quantity and quality have an impact on the system’s overall performance, this step is extremely important. It basically involves gathering data on specific variables. 2) Preparing the Data: Data preprocessing is the next step after data collection. It is a method for turning unstructured data into information that can be used to make a decision. Data cleaning is another name for this operation. 3) Pick a Model: An appropriate technique is selected based on the requirements of the task in order to transform preprocessed data into a model. 4) Get the Model Ready: In ML, supervised learning is used to train a model so that it can make better decisions or make better predictions. 5) Assess the Model: A number of parameters are required for the model to be evaluated. The established goals serve as the basis for the parameters. Additionally, one must document the model’s performance in conjunction with the previous one. 6) Adjusting Parameters: This step may consist of: determining the number of training steps, performance, outcome, learning rate, initialization values, and distribution, among other things. 7) Make Inferences: Predicting some outcome from the test dataset is essential for comparing the developed model to the real world. That model can be used to make additional predictions if the outcome matches those of domain experts or opinions that are closer to it. The following are the fundamental steps for disease detection with ML: 1) Collect patient-specific test data. 2) Attributes that are useful for disease prediction are selected during the feature extraction process. 3) After the selection of attributes, the dataset is selected and processed. 4) Different classification methods, as shown in the diagram, can be used to preprocess a dataset to check how accurate disease predictions are. 5) Different classifiers’ performance is compared to find the best one with the highest accuracy.

Deep Learning is a method of artificial intelligence that creates patterns for higher cognitive processes and imitates the human brain’s functions. In contrast, machine learning techniques required first breaking up a haul statement into distinct parts before integrating their results at the final stage; the Profound Learning strategy’s goal is to disentangle the issue start to finish. There is a lot of interest in deep learning in all areas, but especially in medical image analysis. AANs (artificial neural networks) and deep learning can be distinguished from one another by the variations in a wide variety of hidden layers, as well as their interconnectivity and capacity to produce the appropriate input result. Profound learning is a sort of AI, which is a subset of computerized reasoning. The ability of computers to think and act without human intervention is known as machine learning. Deep learning is the process by which computers learn to think by using brain-like structures.

Previously, a machine-learning technique is used in the standard automated diagnostic method, and a clinical expert manually fetched features from diagnosis reports. However, there were times when it is challenging to extract features from a large dataset. A significant obstacle for deep learning models is the absence of the necessary data. Currently, electronic health records are used in medical research. However, there is no established method for evaluating EHRs, so the accuracy of automated diagnostic procedures may be limited. The model won’t be able to accurately diagnose a disease if the system doesn’t collect accurate data, which makes it hard to show accurate predictions. The authors of this paper came up with an efficient deep learning model that can accurately and quickly identify a variety of diseases to address this issue. A Deep CNN model is typically used to diagnose diseases. The neural system then employs information expansion strategies. The image’s raw information is processed by CNN layer by layer to produce a particular pattern. The first few layers are used to locate the extensive feature set, such as diagonal lines, and the subsequent few layers are used to obtain better details and organize them into sophisticated options. The highest and final layer functions like a typical neural network, and the network becomes completely connected. Then, highly specific features like the illness’s various symptoms are combined, and the prediction of the illness is made. The creators in redressed to disentangle the issue the of lacking data or missing qualities.

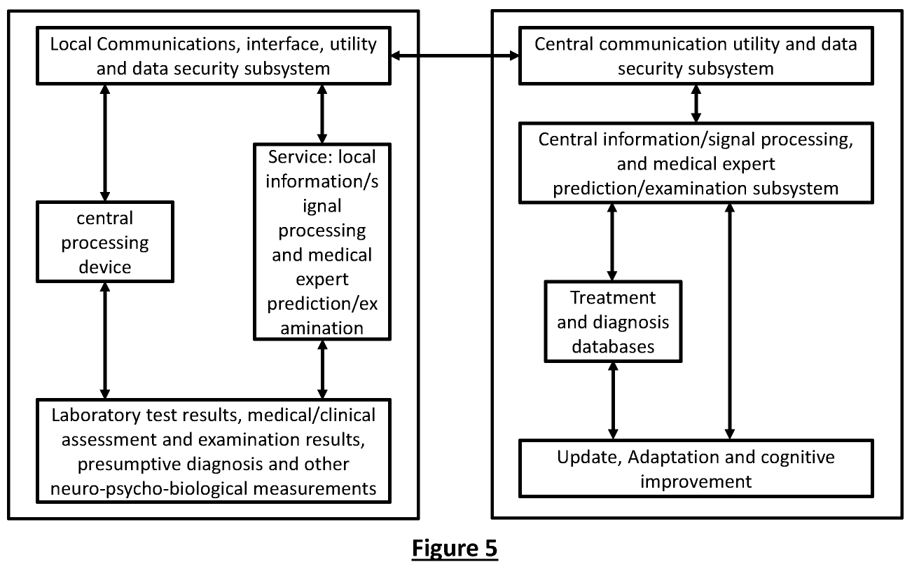

FIG. 5 illustrates a system with remote or central data/signal processing. As much relevant biometric, demographic, neurological, psychological, psychiatric, laboratory, and clinical information as possible about the patient is gathered by the doctor or an assistant. Neuro-psycho-biological indicators such as demographic information, past history, symptom presentation, a list of medical co-morbidities, laboratory results, selected personality and cognitive functioning measures, pharmacogenetic data, and biological data derived from electrophysiological, magnetic, electromagnetic, radiological, optical, infra-red, ultrasonic, acoustic, biochemical, medical imaging, and other investigative procedures and attributes could be included in this information. If it is available, the physician’s presumptive diagnosis is also provided. After that, either a computer technique that has been pre-loaded into the user’s computer or another digital processing device of a similar nature is used to process this data on the spot or it is sent electronically to a remote central processing site. A machine learning and inference method will be used to analyze the data in both cases. A report based on the response-probabilities associated with a variety of potential treatments for the diagnosed condition and, optionally, a list of diagnostic possibilities ranked by likelihood will be produced by this procedure. The physician is then promptly provided with a list of recommended treatments and associated response probabilities, as well as an optional list of diagnostic possibilities ranked by probability or likelihood.

Measures of the functioning and anatomy of the brain and nervous system, such as EEG waveforms, MRI scans, other medical imaging, and various clinical and laboratory assessments, can generate a large set of quantitative values and information for mental and neurological disorders. Even an expert in the field is unable to conduct an effective analysis of this extremely complex dataset. By making use of cutting-edge cognitive signal/information processing techniques as well as computational devices, the present invention offers an intelligent approach to completing this challenging endeavor. The user of this analytical method can (optionally) estimate the diagnosis and divide patients who meet the diagnostic criteria for a specific illness into subgroups that have a preference for one or more treatment options. The current method is a significant advancement in clinical management because it eliminates much of the uncertainty that is present in current clinical practice.

Advanced methods of “signal/information processing” and “machine learning and inference” underpin the present invention’s system and method. This innovation incorporates a computerized robotized clinical master framework equipped for coordinating different arrangements of neurological, mental, mental, natural, segment and other clinical information to improve the viability of the doctor by utilizing AI and surmising strategies to gauge the likelihood of reaction to a scope of treatment prospects fitting for the sickness analyzed, and, alternatively, to give a rundown of symptomatic potential outcomes rank-requested by probability/likelihood. The sign/data handling technique incorporates a few phases including pre-handling, sifting, highlight extraction and element choice, low-layered portrayal, information grouping, measurable and Bayesian demonstrating and examination, mathematical investigation, choice/assessment organizations, building and learning prescient and assessor models utilizing preparing information, and integrating laid out and tried treatment rules and symptomatic order frameworks. The principles and models will be improved by picking up, joining and melding different AI strategies to fabricate a progressive, staggered and organized framework and model that cycles and gathers the information in various levels and handles missed credits.

A capability for adaptive or gradual learning is an essential component of the “medical digital expert system”. The quantity and quality of the training data have a significant impact on the effectiveness of any classification, recognition, or regression process. By continuously acquiring new training data as it becomes available, the system in this invention improves its own performance and reliability. This is achieved by criticism from the family doctor, clinician and additionally quiet to the focal handling site. An estimate of the patient’s reliability as a historian, adherence to treatment, and adequacy of prescribed therapy (e.g., drug dose and duration of administration) are all included in this feedback, which consists of both qualitative and quantitative data describing the patient’s response to the prescribed treatment. The classification/recognition technique’s performance is enhanced by enhancing the computational methods and system for treatment-response-prediction. Only outcome data collected from a dependable historian following an adequate treatment course is added to the training dataset. Optionally, additional information regarding the accuracy of the initial diagnosis as provided by the disclosed diagnostic estimation, detection, and prediction technique will be gathered from the patient’s physician. By this point, the physician will have made additional observations of the patient, including evaluating the efficacy of the prescribed treatment and reviewing new laboratory data. As displayed in FIG. 5, the estimation and prediction models only include valid and reliable data. The clinician is promptly provided with a report that details the likelihood of response to a variety of treatments or therapies that are appropriate for the diagnosed condition and, if desired, a variety of diagnostic possibilities. Even though this system can use the doctor’s estimated diagnosis (when it is available), findings that might point to a different diagnosis than the doctor’s preferred one can be found and sent to the attending doctor.

Even though this system may be beneficial to the family physician as well as the expert specialist, it will be especially useful in situations where expert specialists or family physicians may not be readily available, necessitating the administration of care by other clinically trained personnel such as nurse practitioners or other providers who are not physicians. A patient with access to relevant attributes and information about himself or herself, a laboratory operator, a health professional, a researcher, or an organization seeking to screen out individuals who may be at risk of developing a psychiatric, neurological, or medical illness or condition can be the user of the medical digital expert system in various embodiments, applications, and examples.

In the case of psychiatric illnesses and disorders, for instance, there are numerous potential “indicators” of patient response to treatment. Functional magnetic resonance imaging (fMRI), personality traits, economic and social status, prior psychiatric history, sleep patterns, and other features are among these. Antidepressants like venlafaxine, for instance, may be more effective in patients who have higher metabolic rates in particular brain regions, as shown by fMRI images. Additionally, it has been reported that patients with abnormal sleep EEG profiles have a significantly less favorable clinical response to short-term interpersonal psychotherapy.

Pattern classification or pattern recognition and regression methods, artificial or computational intelligence, data mining, statistical data analysis, computational learning, and cognitive machines, among others, are all examples of machine learning paradigms. are able to classify objects in the same way that a human can. For instance, these techniques can determine whether a particular image best depicts a “nut” or a “bolt.” The image is given its “features,” or attributes, by these techniques. The features are designed to cluster over specific Euclidean space regions according to the object’s class. The collection of training data is a crucial step in any machine learning process. The objects that are presented to the classifier and whose classes are known make up the training data. Because of this, the classifier is able to classify the characteristics, models, and clusters. For instance, when an unclassified object is presented to the classifier in one straightforward manner, its class can be ascertained by locating the cluster that most closely matches the object’s features. When the target variable is continuous, models that perform regression or interpolation can also be built using machine learning.

The aforementioned indicators have previously been used independently to predict a patient’s response to a particular treatment. The present method uses machine learning to classify the predicted patient diagnosis and/or response to a set of given treatments by combining the data from as many indicators and attributes as possible. The utilization of a wide grouping of highlights fundamentally works on the nature of the forecast in contrast with past strategies.

To confirm a diagnosis, estimate a number of diagnostic possibilities, and rank order, according to likelihood of response, a number of treatment options that might be reasonably considered to treat that illness or condition, the present system functions as a digital version of an experienced clinical expert-for example, an expert physician, psychiatrist, or neurologist—who reviews various available information, including neuro-psycho-biological, clinical, laboratory, physical, and pharmacogenetic data and information and evidence.

When the neuro-psycho-biological data for a particular test patient are maximized, the medical digital expert system’s predictive accuracy is ideal. However, in practice, patients do not receive every possible investigation and test because of time, cost, accessibility, or other factors. As a result, the disclosed system is built to function flexibly even when data is missing, provided that the minimum data requirements have been met (for instance, age, sex, and EEG data in psychiatric disorders and illnesses). The expert system analyzes the set of available data and attributes for each patient. The treatment response prediction and, optionally, the diagnostic estimation result will be sent to the doctor electronically. A set of EEG data and a specific set of clinical depression rating scales are, for instance, recorded and entered into the medical digital expert system for a suspected mood disorder in one of its simplest routines. However, by reducing ambiguities and extracting relevant and crucial information that is hidden in various forms of data, measuring more clinical and laboratory data and collecting more laboratory data, neurobiological, psychological, personality, and cognitive attributes and information may assist the expert system and will increase its performance. The disclosed system could also send a prompt to the clinician asking for the results of a specific test, procedure, or other clinical data that could significantly boost the technique’s performance. A reanalysis could then incorporate these new data.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A system for predicting diseases in its early phase using artificial intelligence, the system comprising:
an image acquisition device for collecting medical images in digital format from a plurality of medical prediction centers and a plurality of medical record databases, wherein the collected images are typically captured using one of both of a general-purpose camera or real-time image capturing tools such as CT scan, radiology, MRI, Ultrasound, and nuclear medicine imaging;
an image pre-processing device for enhancing the visual quality of an image by reducing noises and identifying the image’s texture, color, and shape to produce a clean image, wherein the image pre-processing device comprising resizing images to lower pixel resolution to reduce the processing time and cropping images to remove unnecessary area and retaining the area of interest thereby eliminating the noise using filters followed by transforming the original RGB color to grayscale intensity to remove undesired variations in color;
an image segmentation device for extracting the region of interest from the image’s background by identifying each image’s pixel characteristics, and dividing the image into segments consisting of similar characteristic pixels;
a feature extraction and selection device for extracting a set of features selected from Asymmetry index, Entropy, Autocorrelation, Homogeneity, and Contrast used for the classification stage from the region of interest of the image and selecting the optimized features from the set of features;
a model training device for training a fuzzy logic-based prediction model and a plurality of diagnosis-specific treatment response models to predict treatment response using an artificial intelligence and storing in a cloud server platform, wherein the fuzzy logic-based prediction model comprises:
a fuzzifier for converting the medical images input into the fuzzy values;
an inference engine for processing the fuzzy value by the reasoning engine employing a set of rules act as a set of rules to the cognitive content;
a knowledgebase consists of rules, structured and unstructured information also named the database; and
a de-fuzzifier for defuzzification of the fuzzy value upon changing the output from the logical thinking engine into medical images;
a central processing device coupled to a user input device for receiving a subject patient dataset including features obtained for a reduced feature dataset and comparing the subject patient dataset to a feature data scheme for predicting a response for the subject patient, wherein comparing the subject patient dataset comprising determining a subject patient diagnosis of one of the known disorders indicated for the subject patient by the subject patient dataset upon deploying the prediction model to the subject patient dataset and applying the diagnosis-specific treatment response models to the subject patient dataset for predicting the response for the subject patient and predicting the diseases in its early stage, wherein the central processing device is configured to generate a medical report along with severity of the disease and stage of the disease.

2. The system of claim 1, wherein the prediction model is employed for determining a diagnosis of a plurality of known disorders indicated by individual patient dataset and the plurality of diagnosis-specific treatment response models corresponding to a specific diagnosis of the known disorders, the treatment response models configured to use feature data to predict treatment response, wherein the prediction model is configured to:
secure a three-layered ovary picture of a subject through clinical imaging gear, and performing picture denoising and upgrading treatment, wherein the image denoising enhancement process of the biomarker-based ovarian cancer assessment method comprising of completing essential denoising treatment on the first three-layered ovary picture to get an essential denoising picture and calculating the residual quantity of a central pixel for each unit region on the original three-dimensional ovary image using the respective numerical values of the specific energy parameters for the initial denoising image and the three-dimensional ovary image;
compare the image to the position of the ovarian tumor after the enhancement treatment;
utilize a medical instrument to measure the concentration of at least one small molecule biomarker in an ovarian cancer tumor of a subject;
compare a control sample to the concentration of the small molecule biomarker that was obtained, wherein in the event that the convergence of the little sub-atomic biomarkers surpasses or is lower than a relating limit esteem, getting CA125 information, HE4 information and Dad information of a serum test to be distinguished of the subject by utilizing an ID program;
utilize the CA125, HE4, and PA data to calculate an area value under a working characteristic curve; and
use an evaluation program based on the concentration of the small molecule biomarker, the CA125 data, the HE4 data, and the PA data from the serum sample, as well as the area value under the working characteristic curve, evaluating the subject’s ovarian cancer condition and producing an evaluation report for a doctor to diagnose and select a treatment mode.

3. The system of claim 2, wherein the detection of the concentration of at least one small molecule biomarker in the ovarian cancer tumor is accomplished using the biomarker-based ovarian cancer assessment method, wherein the biomarker-based ovarian cancer assessment method comprises:
obtaining a sample from the subject, chosen from the blood, serum, and plasma categories; the little atom biomarker is chosen from the gathering comprising of: hydroxy acids, adipic acid, hydroxybutyric acid, and ketone bodies; dihydroxybutyric acid; trihydroxybutyric acid;
detecting the ovarian cancer-specific small molecule biomarker by contacting the sample with an antibody or antigen-binding fragment that is capable of specifically binding to it;
reading a decile value from the frequency profile of concentrations of the small molecule biomarker and comparing the determined concentration of the small molecule biomarker to the reference frequency profile of concentrations of the small molecule biomarker.

4. The system of claim 1, wherein the image is resized to have a fixed pixel using an image scaling technique such as normalization, and the image’s color space transformation techniques are used to transform the original RGB color to grayscale intensity to remove undesired variations in color, wherein the contrast enhancement technique is used to sharpen the border of the images and improve the brightness between the foreground and background of the image, wherein the degraded image is recovered from a blurred and noisy image in the image restoration, wherein a plurality of filtering techniques are used to de-noise or suppress and smoothen the image, selected from Median filter, Adaptive median filter and to restore the image from blur, which is caused due to the poor focusing of the camera, wherein restoration is performed by using filters preferably a Gaussian filter, wherein the images are smoothened using an image restoration filter, and the image still contain artifacts or other noises, which are removed using various methods such as Curvilinear structure detection, Mathematical morphology, Top Hat transform, Bottom Hat transform, Dull Razor, and Gabor filter.

5. The system of claim 1, further comprises a control unit equipped with the artificial intelligence for generating the feature data scheme, wherein the control unit comprises:
a cloud server for storing a first-level training dataset that contains records with measured patient-related data from a lot of patients, including clinical and/or laboratory data, diagnoses of the presence or absence of known disorders, and information on patient treatment responses, wherein the first-level training dataset further includes one or more of markers are selected from the group comprising following component : Blood Hemoglobin concentration (HbC), transferrins, kreatinin, blood platelet, low-density lipoprotein (LDL), albumin, total protein and calcium; and
a processor for processing the measured patient-related data to extract features using the measured patient-related data to build an extracted feature dataset and generating the feature data scheme by processing the extracted feature dataset thereby processing the data to produce characteristics that seemed to discriminate for an effective prediction, resulting in the reduced feature dataset, wherein the feature data strategy includes a reduced feature dataset with a lower cardinality than the extracted feature dataset, wherein the individual Z score of each marker Mi is determined by following formula, where ME (i, j) is the subject’s individual average value, VAR (i, j) is the subject’s individual variance, and Mi tables show the value of one of the described markers at time i. (2.3) The weighting function is then used to combine each individual Z score, wherein the weighting function is derived from plasma volume, which is the known variation of each relevant marker, and the consistency between all Z scores, wherein the Blood Starch is the estimated value of the capacity variation when using the Z score.

6. The system of claim 5, wherein the processor is configured to cause the system to determine the medical images through the first recognition model to generate the lesion recognition report used for indicating whether the medical images comprises the lesion, the processor is configured to cause the apparatus to search the medical images for a lesion feature by using the artificial intelligence, wherein the lesion feature being a second image feature obtained by learning a first medical image set of a normal organ and a second medical image set of an organ having a lesion by the deep learning network during training to generate the lesion recognition report according to a second searching report, and the lesion feature existing in the second medical image set and not in the first medical image set.

7. The system of claim 6, wherein a feature response of the lesion feature of the first lesion degree in the digital image having a lesion degree lower than the first lesion degree, which is less than a threshold, wherein the lesion degree recognition report of the medical images further comprises a lesion degree label of the medical images and the lesion degree label of the medical images comprises:
a first recognition report of an image block having a severe lesion degree in image blocks segmented from the medical images;
a second recognition report of a lesion degree of the medical images determined using feature information of all the image blocks; and
a comprehensive report determined using the first and second recognition report.

8. The system of claim 1, wherein the stage is preferably defined from 0-5, wherein 0 indicates perfectly fine and 5 is a worst case, that may require serious surgery, wherein the central processing unit, using the artificial intelligence prescribes a treatment plan according to the stage and type of the disease, wherein the diseases includes skin diseases, liver diseases, heart diseases, Alzheimer, cancer and the like, wherein the biomarker-based ovarian cancer assessment method is defined by the fact that an identification procedure is used to obtain the CA125, HE4, and PA data of the subject’s serum sample in the event that a small molecule biomarker selected from the group consisting of hydroxyacids and adipic acid is increased in comparison to a control.

9. The system of claim 8, wherein an exemplary treatment plan, in case cancer, provides a radiotherapy dose distribution upon receiving anatomical data of a human subject and generating a radiotherapy dose data corresponding to the mapping thereby converting the radiotherapy dose data from the generative model into a radiotherapy dose distribution followed by outputting the radiotherapy dose distribution for use in the radiotherapy treatment of the human subject, wherein the anatomical data indicating a mapping of an anatomical area for radiotherapy treatment of the human subject, and wherein the radiotherapy dose data from the generative model identifies radiotherapy dosage to be delivered to the anatomical area.

10. The system of claim 1, wherein the prediction of prostate carcinogenesis and metastasis comprises taking a three-dimensional image of a person’s prostate and bladder and selecting a layer in a sagittal image that passes through the bottom of the bladder thereby obtaining a cross-sectional image at the layer, followed by identifying the fat outline and the prostate outline around the prostate in the cross-sectional image, which calculates the fat area around the prostate (PPFA) based on the area in the fat outline around the prostate, wherein the proportion PPFA/Dad of the region of the fat around the prostate to the region of the prostate, and the gamble worth of the event and the metastasis of the prostate malignant growth is in direct extent to the proportion PPFA/Dad, wherein the central processing device uses a formula based on an age variable, a rectal index variable, a family genetic history variable, a prostate image report and a data system scoring variable, a PSA value variable, and a ratio variable of a peripheral fat area of the prostate and a prostate area to calculate a risk value for the first diagnosis of prostate cancer, wherein the output device then displays the risk value for the first diagnosis of prostate cancer, wherein the formula is as follows:
$$\begin{matrix}\text{Logit} \left(\text{P}\right) \text{=In} \left(\text{P} / \left(\text{1-P}\right)\right) \text{=} \\ \text{1} \text{.037*Age+coefDRE+coefHistory+1} \text{.033*PSA+coefPIRADS+} \\ \text{1} \text{.066*} \left(\text{PPFA} / \text{PA}\right)\end{matrix}$$
.

11. The system of claim 10, wherein the prediction of prostate cancer’s occurrence and metastasis comprises: the handling gadget is utilized for diagnosing lymph hub metastasis probability factors, prostate picture reports and information framework scoring factors, proportion factors of fat region around the prostate and prostate region, Gleason scoring factors, obsessive T stage factors, public service announcement esteem factors and Ki-67 articulation level factors as indicated by X-ray before an activity, working out to get a lymph hub metastasis risk worth of a prostate malignant growth patient as per a recipe, and yielding the lymph hub metastasis risk worth of the prostate disease patient by the result gadget, wherein the equation is as per the following: Logit(P)=In(P/(1-P))=coefPre-LNM+coefPIRADS+coefRatio+coefpT-stage+1.008*PSA+1.152*Ki-67, where P is the predicted value of prostate cancer’s lymph node metastasis risk, coefPre-LNM is the possibility of lymph node metastasis diagnosed prior to MRI surgery, coefPIRADS.

12. The system of claim 10, wherein for the purpose of predicting the occurrence of prostate cancer, an age parameter, a rectal index parameter, a family genetic history parameter, a PSA value parameter, and a PIRADS scoring parameter are combined with the ratio PPFA/PA of the area of the fat surrounding the prostate to the area of the prostate.

13. The system of claim 1, wherein the fuzzy logic-based prediction model involves using the Dopplerographic method to measure quantitative blood flow indicators, wherein the maximum systolic speed and resistance index are assessed at the level of the interlobar renal arteries before and 30 minutes after an intramuscular injection of lasix at a rate of 1 mg/kg and patients with a final diastolic rate decrease of more than 5% and an increase in resistance index of more than 2% are diagnosed with a normal response.

14. The system of claim 1, wherein the fuzzy logic-based prediction model employs at least two types of cancer-related proteins in a sample obtained from a subject having cancer as a prognostic indicator of cancer by identifying at least two types of cancer-associated proteins in the sample from the subject and quantifying the at least two cancer-associated proteins in the sample thereby normalizing the at least two cancer-related proteins in the sample to obtain a normalized value for each cancer-related protein in the sample followed by obtaining a biomarker index and comparing the normalized value of the first cancer-related protein adding a technique, wherein the carcinoma is selected from the group consisting of breast, lung, prostate, colon, liver, thyroid, kidney, and bile duct carcinomas.

15. The system of claim 14, wherein a tumor antigen selected from the following group is present in at least one of the two types of cancer-related proteins: AKT; p-AKT; CA150, Tn antigen in the blood; CA19. -9; CA50; CAB39L; CD22; CD24; CD63; CD66e, CD66a, CD66c, and CD66d; CTAG1B; CTAG2; Antigen oncofetal (CEA); EBAG9; EGFR; FLJ14868; FMNL1; GAGE1; GPA33; LRIG3; lung cancer, group two; MAGE1, M2A tumor fetal antigen MAGEA10; MAGEA11; MAGEA12; MAGEA2; MAGEA4; MAGEB1; MAGEB2; MAGE 3; MAGEB4; MAGEB6; MAGE1; MAGE1; MAGEH1; MAGE2; MGEA5; Protein kinase MOK; MAPK; p-MAPK; mTOR; p-mTOR; MUC16; MUC4; antigen related to melanoma; OCIAD1; OIP5; ovarian malignant growth related antigen; PAGE4; PCNA; PRAME; plastin L; prostate mucin antigen (PMA); antigen specific for prostate (PSA); PTEN; RASD2; ROPN1; SART2; SART3; SPANXB1; SSX5; STEAP4; STK31; TAG72; TEM1; XAGE2; 1-fetoprotein, a Wilms tumor protein; and original tumor antitumor of epithelial origin. The method of claim 1, in which at least one of the two types of cancer-associated proteins includes a tumor-associated antigen from one of the following groups: 5T4; AKT; p-AKT; ACRBP; blood bunch Tn. CD164; CD20; CTHRC1; ErbB2; FATE1; HER2; HER3; GPNMB; Galectin8; HORMAD1; LYK5; MAGEA6; MAGEA8; MAGEA9; MelanA; gp100 melanoma; NYS48; PARP9; PATE; prostein; PTEN; SDCCAG8; SEPT1; SLC45A2; TBC1D2; TRP1; XAGE1, wherein the cancer is selected from the group consisting of Adrenal tumors, bile duct cancer, bladder cancer, bone cancer, brain tumors, breast cancer, heart sarcoma, cervical cancer, colorectal cancer, uterine Endometrial cancer, esophageal cancer, germ cell cancer, gynecological cancer, head and neck cancer, hepatoblastoma, kidney cancer, pharyngeal cancer, leukemia, liver cancer, lung cancer, lymphoma, melanoma, multiple myeloma, neuroblast Cell tumor, oral cancer, ovarian cancer, pancreatic cancer, parathyroid cancer, pituitary tumor, prostate cancer, retinoblastoma, rhabdomyosarcoma, skin cancer (non-melanoma), stomach (digestive organ) cancer, testicular cancer Thyroid cancer, uterine cancer, vaginal cancer, vulvar cancer, and Wilms tumor.

16. The system of claim 14, wherein the artificial intelligence is offered for the cancer-associated protein to serve as a marker for the presence of cancer in the subject upon discovering the presence of a first cancer-related protein in a biological sample taken from the individual, which may be PTEN, p-AKT, p-mTOR, p-MAPK, EGFR, HER2, HER3, or a combination of two or more of these proteins and determining the first cancer-associated protein’s degree of protein expression thereby comparing the first cancer-associated protein’s protein expression level in the biological sample to a predetermined statistically significant cutoff value, where non-cancerous changes in the first cancer-associated protein’s protein expression levels in the biological sample compared to the sample indicate the presence of cancer in the subject.

17. A method for predicting diseases in its early phase using artificial intelligence, the method comprising:
collecting medical images in digital format from a plurality of medical prediction centers and a plurality of medical record databases using an image acquisition device, wherein the collected images are typically captured using one of both of a general-purpose camera or real-time image capturing tools such as CT scan, radiology, MRI, Ultrasound, and nuclear medicine imaging;
enhancing the visual quality of an image by reducing noises and identifying the image’s texture, color, and shape to produce a clean image through an image pre-processing device, wherein the image pre-processing device comprising resizing images to lower pixel resolution to reduce the processing time and cropping images to remove unnecessary area and retaining the area of interest thereby eliminating the noise using filters followed by transforming the original RGB color to grayscale intensity to remove undesired variations in color;
extracting the region of interest from the image’s background by identifying each image’s pixel characteristics, and dividing the image into segments consisting of similar characteristic pixels by employing an image segmentation device;
extracting a set of features selected from Asymmetry index, Entropy, Autocorrelation, Homogeneity, and Contrast used for the classification stage from the region of interest of the image and selecting the optimized features from the set of features using a feature extraction and selection device;
training a fuzzy logic-based prediction model and a plurality of diagnosis-specific treatment response models to predict treatment response using an artificial intelligence and storing in a cloud server platform by deploying a model training device; and
receiving a subject patient dataset including features obtained for a reduced feature dataset via a user input device and comparing the subject patient dataset to a feature data scheme for predicting a response for the subject patient using a central processing device, wherein comparing the subject patient dataset comprising determining a subject patient diagnosis of one of the known disorders indicated for the subject patient by the subject patient dataset upon deploying the prediction model to the subject patient dataset and applying the diagnosis-specific treatment response models to the subject patient dataset for predicting the response for the subject patient and predicting the diseases in its early stage, wherein the central processing device is configured to generate a medical report along with severity of the disease and stage of the disease.

18. The method of claim 17, wherein an in vitro method for diagnosing a patient’s tumor disease using diagnosis-specific treatment response models comprising steps of:
i) finding an IVD marker or IVD marker panel with a relatively high sensitivity to the tumor disease in at least one patient biological sample;
ii) figuring out how many patients tested positive because of a modified reference range for the IVD marker or IVD marker panel, where the modified reference range is one that is adjusted so that a certain number of people who have false negative tests, a certain number of people who have false positive tests, and a certain number of people who will eventually need to be subjected to imaging diagnostics to clarify false negative and false positive results are balanced in relation to one another so that tumor screening may be possible; and
iii) deciding to use an imaging technique specific to the tumor disease so that at least one of the possible false negative and false positive IVD results can be clarified; or performing an imaging technique to image the tumor, or repeating (i) and (ii) after a predetermined time period.

19. The method of claim 18, wherein the biological sample is selected from a blood sample, a serum sample, a plasma sample, a urine sample, a fecal sample, a saliva sample, a spinal fluid sample, a nasal discharge sample, a sputum sample, a bronchoalveolar lavage sample, a semen sample, a breast discharge sample, a wound discharge sample, an ascites sample, a gastric juice sample or a sweat sample.
